(19) United States
(12) Patent Application Publication
(10) Pub. No.: US 2019/0168157 A1
(43) Pub. Date: Jun. 6, 2019

(54) DEVICE AND METHOD FOR PURIFYING GAS IN ENCLOSED SPACE

(71) Applicant: CHINA INNOVATION INSTRUMENT CO., LTD, Ningbo (CN)

(72) Inventors: Luhong WEN, Ningbo (CN); Yang YANG, Ningbo (CN); Zhenzhi SHI, Ningbo (CN); Peng ZHAO, Ningbo (CN); Shundi HU, Ningbo (CN)

(21) Appl. No.: 16/257,327

(22) Filed: Jan. 25, 2019

Related U.S. Application Data
(63) Continuation of application No. PCT/CN2017/109956, filed on Nov. 8, 2017.

(30) Foreign Application Priority Data
Dec. 31, 2016 (CN) 201611268276.3

(51) Int. Cl. B01D 53/79; A61L 2/20; A61L 9/015

(52) U.S. Cl. CPC B01D 53/79; A61L 9/015; A61L 2/20

(57) ABSTRACT

A device for purifying a gas in an enclosed space includes: a container containing a target gas with a content of α, content of the target gas in air being β ranging from 0% to 1%, and α>β; a sensor configured to detect the content of the target gas in the enclosed space; a first valve arranged in a gas path between the container and a first purification unit; the first purification unit with an output end in communication with the enclosed space; a second valve with an input end in communication with the air and an output end in communication with a second purification unit; the second purification unit with an output end in communication with the enclosed space; and a gas path switch module with an input end in communication with the enclosed space. Also, a method for purifying gas in an enclosed space is disclosed.

### CROSS-REFERENCE TO RELATED APPLICATIONS

This application is a continuation of International Patent Application No. PCT/CN2017/109956, filed on Nov. 8, 2017, which claims the benefit of priority from Chinese Application No. 201611268276.3, filed on Dec. 31, 2016. The contents of the aforementioned applications, including any intervening amendments thereto, are incorporated herein by reference.

### TECHNICAL FIELD

The present invention relates to cell culture, and in particular to a device for purifying gas in an enclosed space and a method for controlling the same.

### BACKGROUND OF THE PRESENT INVENTION

Purge dilution in an enclosed space is to decrease the number of particles and microorganisms in the space so that the incoming objects will not contaminate the internal space to maintain a stable sterile environment.

Currently and generally, oxygen is fed by a pure oxygen source to increase the oxygen content in a cabinet to an upper limit, and nitrogen is fed by a pure nitrogen source to decrease the oxygen content in the cabinet to a lower limit, where the upper and lower limits of the oxygen content are set to have a filtration efficiency of 10% or more in a cycle; oxygen is then fed again by the pure oxygen source to increase the oxygen content in the cabinet, called a cycle. The number of contaminants may be decreased to 10⁻⁶ after six cycles, which meets GMP A-grade sterile requirement. However, there exist many disadvantages in such process.

1. Pure oxygen sources and pure nitrogen sources are used to regulate the content of oxygen in the cabinet, resulting in high cost.

2. The decrease of oxygen content requires more gas consumption and higher cost than the increase of oxygen content. Furthermore, the cost increases with the increase of the volume of the enclosed space.

### SUMMARY OF THE PRESENT INVENTION

In order to overcome the deficiencies in the prior art, the present invention provides a device for purifying gas in an enclosed space with low gas consumption and low cost.

The device for purifying gas in an enclosed space comprises:

a container containing a target gas with a content of α, wherein content of the target gas in air is β ranging from 0% to 1%, and α>β;

a sensor configured to detect a content of the target gas in the enclosed space;

a first valve arranged in a gas path between the container and a first purification unit;

the first purification unit with an output end in communication with the enclosed space;

a second valve with an input end in communication with the air and an output end in communication with a second purification unit;

the second purification unit with an output end in communication with the enclosed space; and

a gas path switch module with an input end in communication with the enclosed space.

Another object of the present invention is to provide a method for purifying a gas in an enclosed space, including:

(A1) injecting a purified target gas with a content of α into the enclosed space to increase a content γ of the target gas in the enclosed space; wherein content of the target gas in air is β ranging from 0% to 1%, and α>β;

(A2) stopping injecting the target gas when the content γ of the target gas reaches an upper limit;

(A3) injecting a purified air into the enclosed space to decrease the content γ of the target gas in the enclosed space; and

(A4) stopping injecting the purified air when the content γ of the target gas reaches a lower limit.

Compared to the prior art, the present invention provides the following beneficial effects.

1. Low gas consumption. Content of the target gas used herein is very low in the air, for example, a gas which is not present in the air, or a gas with a content of less than 1% in the air. The content of the target gas in the enclosed space may be adjusted by a target gas of a higher concentration or the air in the environment that is infinite, leading to low consumption of the target gas.

2. Low operation cost. The operating cost is significantly reduced due to the lower consumption of the target gas in each cycle.

### BRIEF DESCRIPTION OF THE DRAWINGS

The disclosure of the present invention will become more apparent with reference to the drawings. It should be understood by those skilled in the art that these drawings are merely for illustrative of the present invention, and are not intended to limit the scope of the present invention.

FIG. 1 is a schematic diagram of a device for purifying gas in an enclosed space according to an embodiment of the present invention; and

FIG. 2 is a schematic diagram of the device for purifying gas in an enclosed space according to an embodiment of the present invention.

### DETAILED DESCRIPTION OF THE PRESENT INVENTION

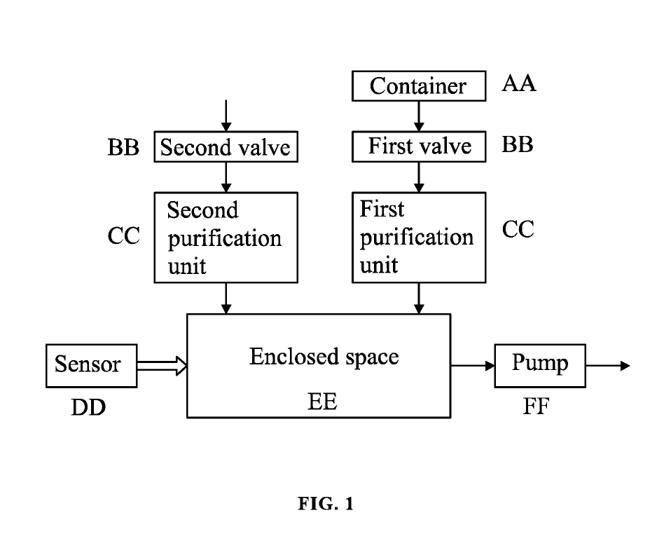

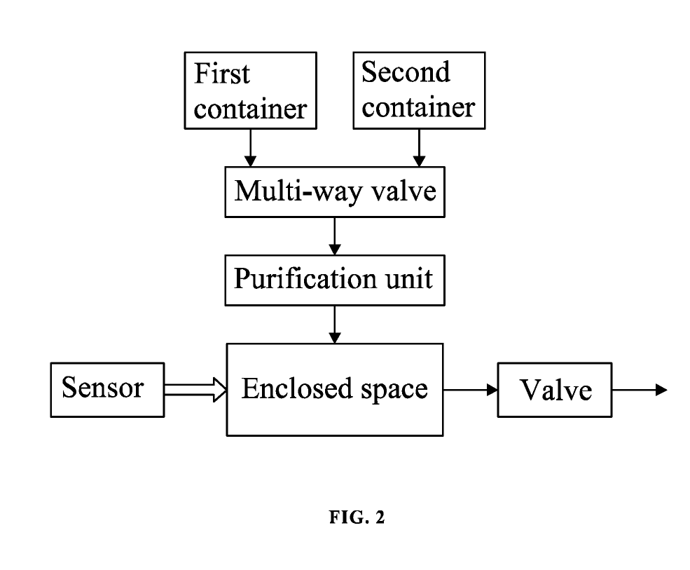

FIGS. 1-2 and the following description describe optional embodiments of the present invention to teach those skilled in the art how to implement and reproduce the present invention. To teach the technical solutions of the present invention, some conventional aspects have been simplified or omitted. It should be understood by those skilled in the art that variants or substitutions derived from these embodiments shall fall within the scope of the present invention. It should be understood by those skilled in the art that the following features can be combined in various ways to form a plurality of variants of the present invention. Therefore, the present invention is not limited to the following optional embodiments, but only defined by the appended claims and equivalents thereof.

### EXAMPLE 1

FIG. 1 schematically shows a device for purifying a gas in an enclosed space according to an embodiment of the present invention. As shown in FIG. 1, the device for purifying a gas in an enclosed space includes:

a container containing a target gas with a content of α (e.g., carbon dioxide with a content of 20%), where content of the target gas in air is β ranging from 0% to 1%, and α>β;
a sensor configured to detect the content of the target gas in the enclosed space;

a first valve arranged in a gas path between the container and a first purification unit;

the first purification unit with an output end in communication with the enclosed space to remove particles and microorganisms in the gas;

a second valve with an input end in communication with the atmospheric environment and an output end in communication with a second purification unit;

the second purification unit with an output end in communication with the enclosed space to remove particles and microorganisms in the gas; and

a gas path switch module, for example, a pump, with an input end in communication with the enclosed space.

This embodiment of the present invention provides a method for purifying a gas in an enclosed space, i.e., a working process of the above purifying device, which includes the following steps.

(A1) The first valve is opened. A target gas with a content of α in a container is injected into an enclosed space through a first purification unit and then is discharged from the enclosed space via a pump to increase a content γ of the target gas in the enclosed space. Content of the target gas in the air is β ranging from 0% to 1%, and α>β. The content of the target gas in the enclosed space is detected by a sensor.

(A2) The first valve is closed to stop the injection of the target gas when the content γ of the target gas reaches an upper limit.

(A3) The pressure in the enclosed space is reduced by the pump. The second valve is opened, and the air is injected into the enclosed space through a second purification unit and gas in the enclosed space is discharged via the pump to decrease the content γ of the target gas in the enclosed space.

(A4) The second valve is closed to stop the injection of air when the content γ of the target gas reaches a lower limit.

The above steps are repeated several times to reduce the number of particles and microorganisms in the enclosed space to meet the GMP A-grade cleanliness requirement. The number of contaminants is also reduced to a level of 10⁻⁶.

### EXAMPLE 2

FIG. 2 schematically shows a device for purifying gas in an enclosed space according to an embodiment of the present invention. As shown in FIG. 2, the device for purifying a gas in an enclosed space includes:

a first container containing a target gas with a content of α, for example, a target gas which is not present in the air or a gas (such as hydrogen) with a content of 1% in the air, where the content of the target gas in the air is β ranging from 0% to 1%, and α>β, and the pressure of the target gas in the first container is higher than the pressure of the gas in the enclosed space;

a second container containing a compressed air, where the pressure of the compressed air in the second container is higher than the pressure of the gas in the enclosed space;

a sensor configured to detect the content of the target gas in the enclosed space;

a first valve arranged in a gas path between the first container and a first purification unit;

the first purification unit with an output end in communication with the enclosed space to remove particles and microorganisms in the gas;

a second purification unit with an output end in communication with the enclosed space to remove particles and microorganisms in the gas, where the first purification unit and the second purification unit are the same purification unit;

a second valve with an input end in communication with the second container and an output end in communication with the second purification unit, where the first valve and the second valve are integrated into a multi-way valve to enable the purification unit to communicate with the first container or the second container; and

a gas path switch module, for example, a third valve with an input end in communication with the enclosed space.

This embodiment of the present invention provides a method for purifying gas in an enclosed space, i.e., a working process of the purifying device, which includes the following steps.

(A1) The multi-way valve is switched to allow a target gas with a content of α in the first container to be injected into the enclosed space through the purification unit and discharged from the enclosed space so as to increase the content γ of the target gas in the enclosed space. Content of the target gas in air is β ranging from 0% to 1%, and α>β. The content of the target gas in the enclosed space is detected by a sensor.

(A2) The multi-way valve is switched to stop the injection of the target gas when the content γ of the target gas reaches an upper limit.

(A3) The multi-way valve is switched to allow a compressed air in the second container to be injected into the enclosed space through the purification unit and discharged from the enclosed space so as to decrease the content γ of the target gas in the enclosed space.

(A4) The multi-way valve is switched to stop the injection of air when the content γ of the target gas reaches a lower limit.

The above steps are repeated several times to reduce the number of particles and microorganisms in the enclosed space to meet the GMP A-grade cleanliness requirement. The number of contaminants is also reduced to a level of 10⁻⁶.

What is claimed is:

1. A device for purifying a gas in an enclosed space, comprising:
a container containing a target gas with a content of α, wherein content of the target gas in air is β ranging from 0% to 1%, and α>β;
a sensor configured to detect a content of the target gas in the enclosed space;
a first valve arranged in a gas path between the container and a first purification unit;
the first purification unit with an output end in communication with the enclosed space;
a second valve with an input end in communication with the air and an output end in communication with a second purification unit;
the second purification unit with an output end in communication with the enclosed space; and
a gas path switch module with an input end in communication with the enclosed space.

2. The device of claim 1, wherein the gas path switch module is a pump with an input end in communication with the enclosed space, and the input end of the second valve is in communication with an atmospheric environment.

3. The device of claim 1, wherein the first purification unit and the second purification are integrated into a purification unit, and the first valve and the second valve are integrated into a switching unit that enables the purification units to communicate with an external space or the container.

4. The device of claim 1, wherein the first purification unit and the second purification unit removes particulates and microorganisms in the gas.

5. The device of claim 1, wherein the input end of the second valve is in communication with an additional container containing a compressed air.

6. A method for purifying a gas in an enclosed space, comprising:
(A1) injecting a purified target gas with a content of α into the enclosed space to increase a content γ of the target gas in the enclosed space; wherein content of the target gas in air is β ranging from 0% to 1%; and α>β;
(A2) stopping injecting the target gas when the content γ of the target gas reaches an upper limit;
(A3) injecting a purified air into the enclosed space to decrease the content γ of the target gas in the enclosed space; and
(A4) stopping injecting the purified air when the content γ of the target gas reaches a lower limit.

7. The method of claim 6, wherein in step (A3), air in an atmospheric environment after purification or a compressed air after purification is injected into the enclosed space to reduce pressure in the enclosed space.

8. The method of claim 6, wherein in step (A1) or (A3), gas in the enclosed space is discharged during the injection of the target gas or the purified air.
